(19) United States
(12) Patent Application Publication
(10) Pub. No.: US 2024/0326379 A1
(43) Pub. Date: Oct. 3, 2024

(54) DUAL LAYER HEAT SHRINK TUBING

(71) Applicant: Zeus Company LLC, ORANGEBURG, SC (US)

(72) Inventors: Cameron Hunter, St. Matthews, SC (US); John Richard Campanelli, West Columbia, SC (US); Zeth Eberling, Lexington, SC (US)

(21) Appl. No.: 18/669,106

(22) Filed: May 20, 2024

Related U.S. Application Data
(63) Continuation-in-part of application No. 17/332,490, filed on May 27, 2021, now Pat. No. 12,005,627.
(60) Provisional application No. 63/030,948, filed on May 28, 2020.

(51) Int. Cl. B32B 7/028; A61L 29/04; A61M 25/00; B32B 1/08; B32B 27/08; B32B 27/28; B32B 27/30; B32B 27/32; B32B 27/34

(52) U.S. Cl. CPC B32B 7/028; A61L 29/049; A61M 25/0045; B32B 1/08; B32B 27/08; B32B 27/285; B32B 27/304; B32B 27/322; B32B 27/34; B32B 2250/24; B32B 2307/536; B32B 2307/736; B32B 2535/00; B32B 2597/00

(57) ABSTRACT

The present disclosure provides a dual layer heat shrink tube having: an inner polymeric layer and an outer polymeric layer. The disclosure further provides associated methods for preparing and using such tubes, as well as to products comprising such tubes.

### CROSS-REFERENCE TO RELATED APPLICATION

The present application is a continuation-in-part of and claims priority to U.S. patent application Ser. No. 17/332,490, filed May 27, 2021, which claims priority to U.S. Provisional Patent Application No. 63,030,948, filed May 28, 2020. The contents of each of these applications are incorporated by reference herein in their entireties.

### FIELD OF THE INVENTION

The present application relates generally to heat shrink tubes, and to methods of producing and using such heat shrink tubes.

### BACKGROUND

Heat shrink tubing generally comprises a plastic material that is extruded into a tubular form and expanded. The extruded and expanded tube is designed to shrink (i.e., decrease in diameter) when heated to a given temperature. As such, heat shrink tubing can serve various functions. It can provide a tight, protective jacketing to closely cover and insulate various elements (e.g., to protect them from abrasion and to provide thermal, chemical, moisture, and/or electrical insulation); it can serve to bundle certain elements together (i.e., within the same heat shrink tube); it can serve to seal/isolate certain elements from others; it can be used to join/fuse two elements, e.g., two tubes together; and it can serve to modify the properties of an underlying material (e.g., by closing around another material and shrinking that material as well). These capabilities render the tubing useful for various purposes and heat shrink tubing finds use across various fields, e.g., environmental, medical, chemical, electrical, optical, electronic, aerospace, automotive, and telecommunications fields. In the medical context, heat shrink tubing is particularly beneficial in designing increasingly small and more complex devices to be inserted into the body (e.g., catheters, endoscopes, etc.).

The suitability of a heat shrink tubing for any given application is dependent, at least in part, on the physical properties of the tubing, particularly after it has been subjected to heat. In particular, a dual layer heat shrink tubing comprises an outer heat shrink tubing/layer that advantageously contracts around a melting inner tubing/layer that flows upon the application of heat to adequately encapsulate the underlying component(s). Any voids or irregularities in the flowed polymeric heat shrink material or that arise upon cooling of the heat shrink material around the underlying component(s) may render the heat shrink tubing ineffective for its intended purpose. If the inner tube/layer melts early relative to the recovering tube/layer, or if the recovery force of the outer tube/layer is too high, the polymer flow upon heating the tubing will lead to an unacceptable increase in recovered tube length as well as overflow of the molten polymer out of the tube ends. If the recovery of the outer tube/layer occurs while the polymer of the inner tube is not sufficiently deformable, or if the recovery force of the outer tube/layer is too low, the underlying component may not be properly encapsulated.

It would be advantageous to provide alternative heat shrink tubing designs to ensure for adequate encapsulation of underlying component(s) and methods of ensuring that heat shrink tubings will function as desired, e.g., providing adequate encapsulation of underlying component(s).

### SUMMARY

The disclosure provides composite heat shrink tubings, also referred to herein as “dual layer heat shrink tubings), which comprise a first, inner tube/layer within a second, outer tube/layer (i.e., a “tube-within-a-tube” structure). For purposes of this disclosure, the inner portion is referred to as the “inner tube,” “inner polymeric tube,” “inner layer,” or “inner polymeric layer.” Likewise, the outer portion is referred to as the expanded or oriented “outer tube,” “outer polymeric tube,” “outer layer,” or “outer polymeric layer.”

The first and second tubes/layers can, in some embodiments, be designed and uniquely evaluated on the basis of certain parameters to ensure that when deployed as a dual layer heat shrink tubing, the first, inner layer will melt and flow under heat while the second, outer layer contracts (serving as an effective heat shrink tubing). This design results in suitable encapsulation and protection of an underlying component or components upon the application of heat. This design can further allow for control over the change in length of the tube, overflow of the first, inner layer and presence of voids during recovery. Advantageously, certain dual layer heat shrink tubings prepared as described herein and recovered under the conditions stated can exhibit optimal encapsulation properties (few voids, little overflow) with an increase in recovered length of the outer layer less than about 20% of its original length.

In some embodiments, the disclosure relates to heat shrinkable polymeric tubes composed of two or more distinct layers comprising one or more polymers that are not cross-linked. In some embodiments, the disclosure relates to multilayer heat shrinkable (“heat shrink”) tubes comprising an outer layer of poly (ether-block-amide) copolymer (PEBA), wherein the PEBA is not crosslinked, and an inner layer of maleated polyethylene (PE). Typically, such multilayer heat shrink tubes are in the form of extruded and expanded tubes (e.g., where each layer of the multilayer has been expanded, e.g., together). It has been found that certain such extruded multilayer tubes can be expanded to form multilayer heat shrink tubes without a crosslinking step. Such multilayer heat shrink tubes can be subsequently employed during catheter shaft assembly processes as an outer jacket for catheter shafts without the need for expensive single-use manufacturing aids such as FEP heat shrink tubes. With propitious conditions of expansion, recovery ratio, and time/temperature profile for the recovery process, a satisfactory catheter shaft can be manufactured using a tube comprising non-crosslinked PEBA lined with maleated PE, which does not require removal of any single-use manufacturing aid prior to use. Several catheter shaft configurations comprising a variety of components can be assembled utilizing such a multilayer heat shrink tube as an outer jacket under conditions to be explained more fully hereafter.

The present disclosure includes, without limitation, the following embodiments.

Embodiment 1: A multilayer heat shrink tubing comprising: an outer layer comprising non-crosslinked PEBA; and an inner layer comprising maleated polyolefin, wherein the maleated polyolefin has a melt temperature that is lower than the melt temperature of the outer layer.

Embodiment 2: The multilayer heat shrink tubing Embodiment 1, wherein the heat shrink tubing has a recovery ratio (RR) greater than about 1.05:1.

Embodiment 3: The multilayer heat shrink tubing of Embodiment 1 or 2, wherein the outer layer consists essentially of the non-crosslinked PEBA.

Embodiment 4: The multilayer heat shrink tubing of any of Embodiments 1-3, wherein the inner layer consists essentially of the maleated polyolefin.

Embodiment 5: The multilayer heat shrink tubing of any of Embodiments 1-4, wherein the maleated polyolefin comprises one or more of maleic anhydride grafted polyethylene, maleic anhydride grafted polypropylene, and maleic anhydride grafted ethylene vinyl acetate copolymer.

Embodiment 6: The multilayer heat shrink tubing of any of Embodiments 1-5, wherein the maleated polyolefin comprises maleic anhydride grafted linear low density polyethylene.

Embodiment 7: The multilayer heat shrink tubing of any of Embodiments 1-6, wherein the weight ratio of the outer layer to the inner layer is between 99:1 and 1:99.

Embodiment 8: The multilayer heat shrink tubing of any of Embodiments 1-6, wherein the weight ratio of the outer layer to the inner layer is between 95:5 and 5:95.

Embodiment 9: The multilayer heat shrink tubing of any of Embodiments 1-6, wherein the weight ratio of the outer layer to the inner layer is between 90:10 and 10:90.

Embodiment 10: The multilayer heat shrink tubing of any of Embodiments 1-6, wherein the weight ratio of the outer layer to the inner layer is between 75:25 and 25:75.

Embodiment 11: The multilayer heat shrink tubing of any of Embodiments 1-10, wherein the RR is greater than about 1.10:1 and/or is reducible in ID by about 9.1%.

Embodiment 12: The multilayer heat shrink tubing of any of Embodiments 1-11, wherein the RR is greater than about 1.2:1 and/or reducible in ID by about 16.7%.

Embodiment 13: The multilayer heat shrink tubing of any of Embodiments 1-12, wherein the RR is greater than about 1.3:1 and/or reducible in ID by about 23.1%.

Embodiment 14: The multilayer heat shrink tubing of any of Embodiments 1-13, wherein the RR is greater than about 1.4:1 and/or reducible in ID by about 28.6%.

Embodiment 15; The multilayer heat shrink tubing of any of Embodiments 1-14, wherein the RR is greater than about 1.5:1 and/or reducible in ID by about 33.3%.

Embodiment 16: The multilayer heat shrink tubing of any of Embodiments 1-15, wherein the RR is greater than about 1.6:1 and/or reducible in ID by about 37.5%.

Embodiment 17: The multilayer heat shrink tubing of any of Embodiments 1-16, wherein a durometer hardness measurement of the outer layer of a flat specimen fabricated by melt pressing the heat shrink tubing in expanded form is about 20 to 80 Shore D.

Embodiment 18: The multilayer heat shrink tubing of any of Embodiments 1-17, wherein the outer polymeric layer is removable after being heat shrunk.

Embodiment 19: The multilayer heat shrink tubing of any of Embodiments 1-18, wherein one or both of the inner layer and the outer layer comprises a filler.

Embodiment 20: The multilayer heat shrink tubing of any of Embodiments 1-19, wherein one or both of the inner layer and the outer layer comprises a blend of polymers.

Embodiment 21: A shrunken multilayer tube, prepared by heating the multilayer heat shrink tubing of any of Embodiments 1-20 such that the inner layer melts, forming an inner encapsulating layer and the outer layer contracts, forming an outer, contracted polymeric layer.

Embodiment 22: The shrunken multilayer tube of Embodiment 21, wherein the outer, contracted polymeric layer is removable.

Embodiment 23: An encapsulated component, comprising a component within the shrunken multilayer tubing of any of Embodiments 21-22.

Embodiment 24: The encapsulated component of Embodiment 23, wherein the component is completely encapsulated by the inner encapsulating layer.

Embodiment 25: A method of encapsulating a component, comprising: applying the multilayer heat shrink tubing of any of Embodiments 1-20 around the component, heating the multilayer heat shrink tubing, and cooling the resulting encapsulated component.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWINGS

In order to provide an understanding of embodiments of the invention, reference is made to the appended drawings, which are not necessarily drawn to scale, and in which reference numerals refer to components of exemplary embodiments of the invention. The drawings are exemplary only, and should not be construed as limiting the invention.

FIG. 1 is a general drawing of a portion of a dual layer heat shrink tubing 10 according to certain embodiments of the disclosure;

FIG. 2 is a schematic drawing of a non-limiting method of expanding polymeric tube 11 to give expanded tube 12 (shown in cross-sectional view);

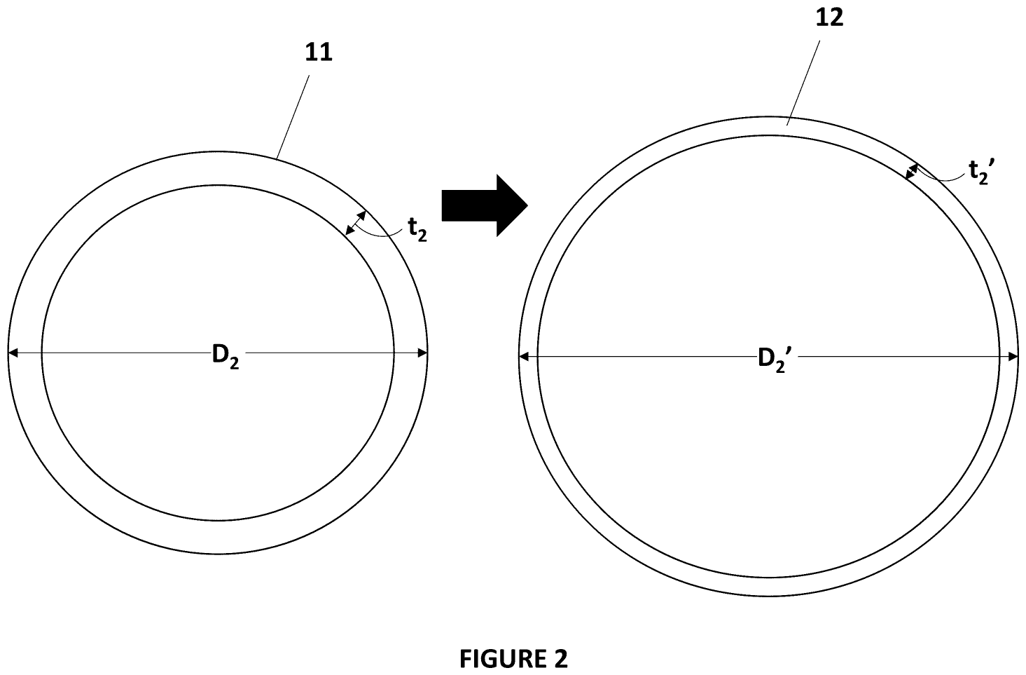

FIG. 3 is a schematic drawing of a non-limiting dual layer heat shrink tubing 10 (shown in cross-sectional view) according to certain embodiments of the disclosure;

FIG. 4 is a general schematic of a process according to certain embodiments of the present disclosure;

FIG. 5 is a plot of DMA temperature sweep of a dual-layer tube of Comparative Example 1 with calculation of tan δ area;

FIG. 6 is a plot of storage modulus versus temperature from a DMA sweep of a dual layer heat shrink tube from Example 1;

FIG. 7 is a plot of DMA temperature sweep of a dual-layer tube of Example 4 with calculation of tan δ area from 235° C. to 323° C.;

FIG. 8 is a schematic of a non-limiting catheter according to certain embodiments of the present disclosure with a corresponding expanded schematic of one cross-sectional end face of the catheter;

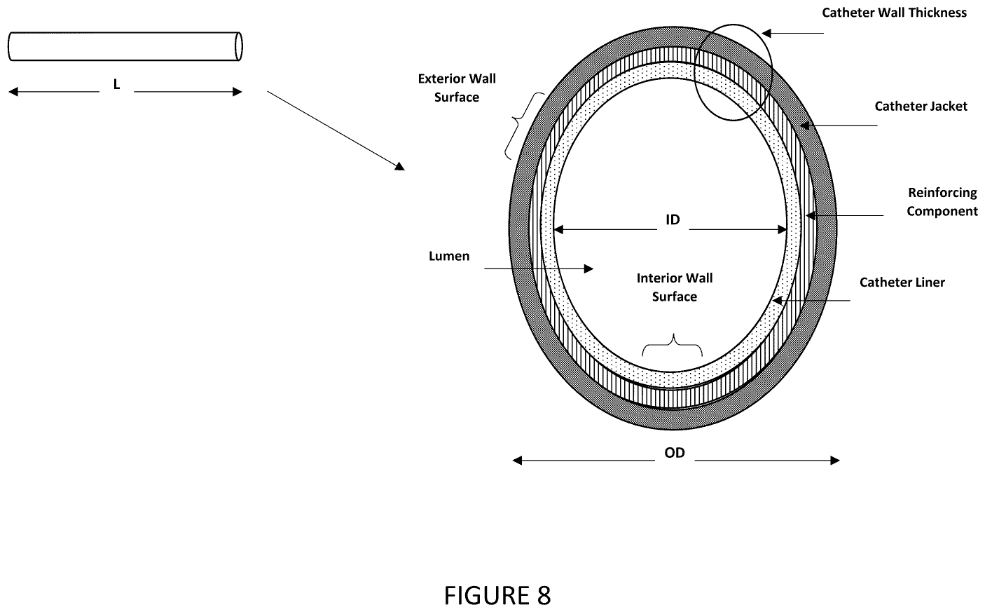

FIG. 9 is a schematic of a non-limiting 2-layer multilayer tube according to certain embodiments of the present disclosure;

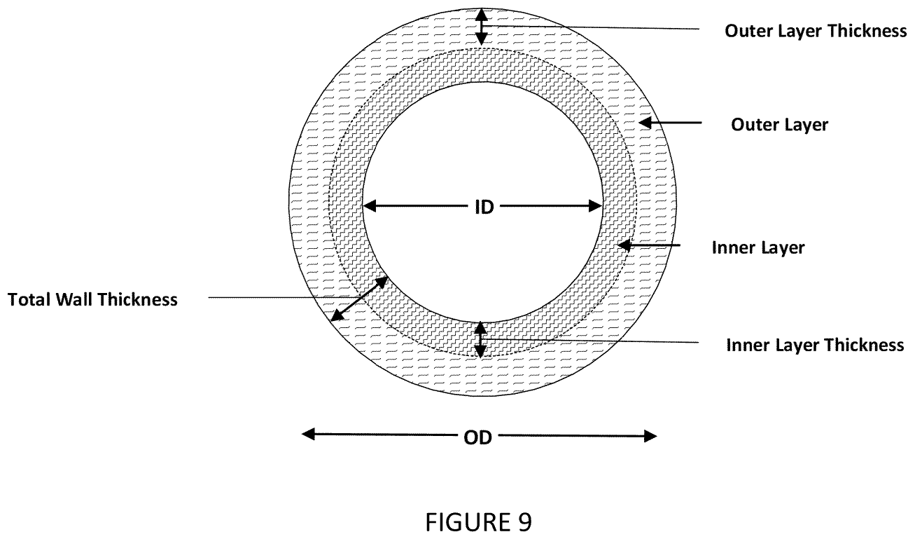

FIG. 10 is a schematic of a non-limiting 3-layer multilayer tube according to certain embodiments of the present disclosure;

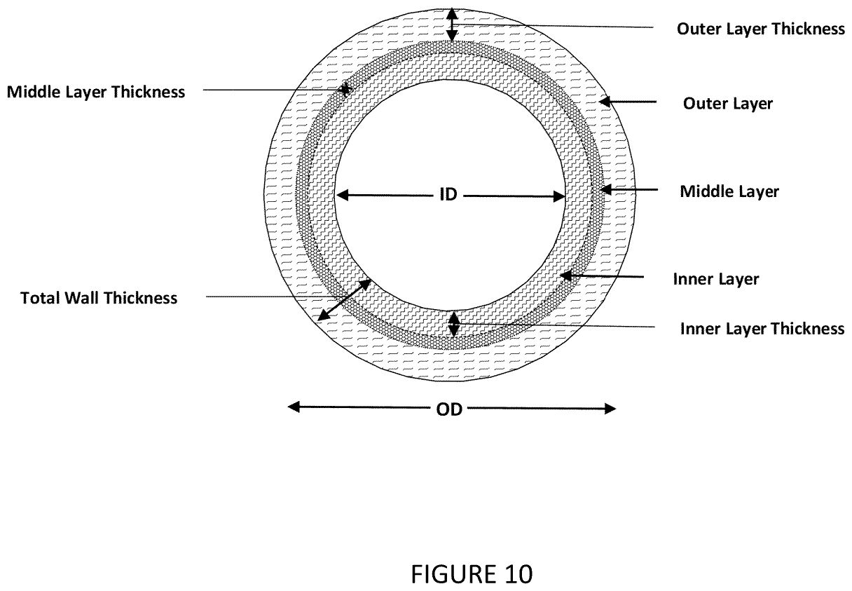

FIG. 11 is a cross section image of mounted Example 2 (30× Magnification);

FIG. 12 is a cross section image of mounted Example 2 (250× Magnification); and

FIG. 13 is a cross section image of mounted Example 2 (250× Magnification).

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present disclosure provides multilayer (e.g., dual) layer heat shrink tubings, comprising a first, inner layer and a second, outer layer. In some embodiments, the first, inner layer of the dual layer heat shrink tubings melts and flows at an elevated temperature, while the second, outer layer contracts, thereby encapsulating an underlying component or part. The inventors of the present disclosure have developed a systematic method of formulating a dual shrink tubing and evaluating a tubing prior to its deployment in a heat shrink capacity (i.e., before it is “recovered”). In particular, by considering the chemical composition and physical characteristics of both the inner layer and the outer layer, a multilayer tube (e.g., dual layer tube) can be provided which exhibits good encapsulation capabilities.

Various features affect the ability of a dual layer tube to exhibit good encapsulation capabilities, e.g., including but not limited to, the polymer flow of the inner layer leading to an unacceptable increase in recovered tube length (e.g., greater than 20% the original tube length) if the inner layer melts early relative to the recovering/outer layer, or if the recovery force of the outer layer is too high; or the underlying component not being properly encapsulated if the recovery of the outer tube occurs while the polymer of the inner tube is not sufficiently deformable, or if the recovery force of the outer layer is too low. Advantageously, the methods and tubings provided herein account for melt/flow and recovery characteristics of both layers in a non-isothermal environment, such that the tubings are provided that can exhibit excellent physical properties and functional properties in heat-shrink applications.

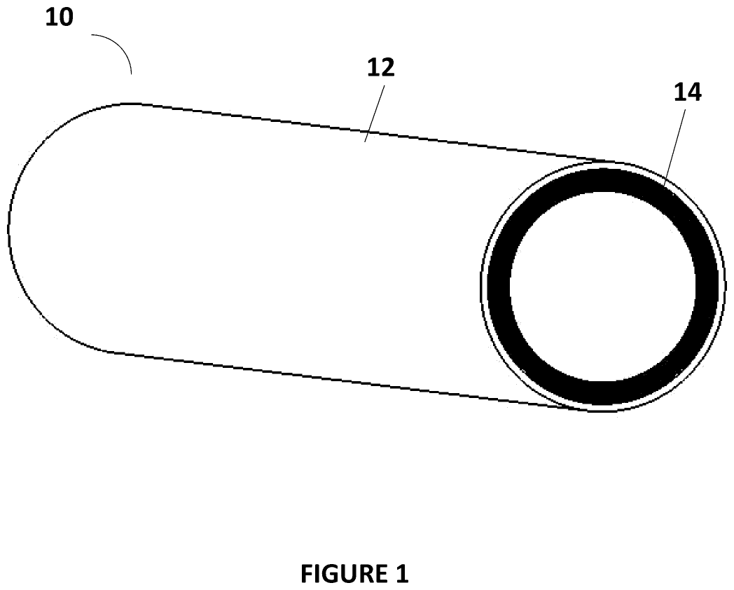

A general schematic of a dual layer heat shrink tubing is provided in FIG. 1. As shown therein, heat shrink tubing 10 comprises an outer, expanded layer 12 and an inner layer 14. At least a portion of the outer surface of inner layer 14 is typically in contact with at least a portion of the inner surface of outer, expanded layer 12. In certain embodiments, substantially the entirety (including the entirety) of the outer surface of inner layer 14 is typically in contact with at least a portion of the inner surface of outer, expanded layer 12. Advantageously, in some embodiments, the layers are in direct contact with one another (e.g., along the full length of dual layer heat shrink tubing 10, with no space therebetween. As will be more evident based on the following discussion, inner layer 14 may be in expanded or unexpanded form.

The overall size of the dual layer heat shrink tubing 10 can vary; the principles described herein are applicable to tubings of a wide range of sizes. The parameters of multilayer heat shrink tubes within the scope of this disclosure (e.g., length, diameter (i.e., expanded inner diameter, ID), and average wall thickness) are not particularly limited. For example, the length of tubes described herein can vary from individually sized units (e.g., in some embodiments, on the order of 0.1 inches to 120 inches for catheter or medical device component manufacturing) to lengths that can readily be transported and further cut into individually sized units to large-scale production lengths (e.g., on the order of hundreds of feet and the like). The diameters of tubes described herein can vary, in particular, depending upon the application for which the tubing is intended. In particular embodiments, the overall diameter of the multilayer heat shrink tubings provided herein (before recovery) range from about 2 mm to about 20 mm, e.g., about 4 mm to about 10 mm. Certain expanded IDs of tubes in certain embodiments described herein, particularly for catheter and medical device uses, can range from about 0.005 inches to about 1.5 inches (e.g., about 0.01 inches to about 0.7 inches or about 0.015 inches to about 0.5 inches), although tubes having expanded IDs outside this range are also encompassed by the present disclosure, particularly in the context of applications in different fields. The layer ratios of multilayer tubes described herein can vary, in particular, depending upon the application for which the multilayer tubing is intended. Certain layer ratios of the multilayer tubes described herein, particularly for catheter and medical device uses, range from 99:1 to 1:99 inches (e.g., 95:5 to 5:95 or 75:25 to 25:75 or 50:50), although multilayer tubes having layer ratios outside this range are also encompassed by the present disclosure, particularly in the context of applications in different fields.

It is noted that, although FIG. 1 depicts a dual (2)-layered heat shrink tubing, the disclosure is not limited thereto; heat shrink tubings with two or more layers, three or more layers, four or more layers, and further layers are also encompassed by the present disclosure. As such, references to heat shrink tubings 10 are understood to encompass multilayered heat shrink tubings with two or more layers (and are not limited to dual layer heat shrink tubings as shown). Thus, although in some embodiments, dual layer tubes are exemplified, the disclosure is not intended to be limited thereto and may encompass tubes with more layers (which can be the same as or different than the inner and/or outer layers as described herein).

Outer, expanded layer 12 of the heat shrink tubing 10 is an expanded (e.g., oriented) heat shrinkable tube. For example, it can be an extruded tube that has been expanded, as outlined below (either expanded alone before combination with inner layer 14 or expanded together with inner layer 14). Outer, expanded layer 12 generally comprises a polymer or polymer blend that is capable of crystallizing or being cross-linked and then expanded radially to a larger diameter (which is then maintained by quenching the tube). In certain embodiments, outer, expanded layer 12 comprises a single type of polymer; in other embodiments, outer, expanded layer 12 comprises two or more of the below-referenced polymers (e.g., in the form of a copolymer or blend). In some embodiments, outer, expanded layer 12 can further comprise one or more additives, e.g., pigments, dyes, fillers, and the like. Certain non-limiting examples of pigments, dyes, and fillers include, e.g., graphene, carbon nanotubes, clays, organoclays, silica, silicates, zeolites, and functional additives to promote slip, impart color, impart conductivity, or provide antimicrobial characteristics.

Suitable polymers for layer 12 include thermoplastic materials. Layer 12 can comprise, e.g., a fluoropolymer and, in particular, a thermoplastic melt-processable fluoropolymer. Suitable fluoropolymers are known and include, but are not limited to, poly (tetrafluoroethylene) (PTFE), fluorinated ethylene propylene (FEP), polyvinylidene fluoride, perfluoroalkoxy alkane (PFA), perfluoro (alkyl vinyl ethers) (PAVE), a tetrafluoroethylene, hexafluoropropylene, vinylidine fluoride terpolymer (THV), polyvinylidene difluoride (PVDF), poly (ethylene-co-tetrafluoroethylene) (ETFE), ethylene chlorotrifluoroethylene (ECTFE), polychlorotrifluoroethylene (PCTFE), polytetrafluoroethylene (PTFE), ethylene, tetrafluoroethylene, and hexafluoropropylene (EFEP), tetrafluoroethylene and perfluoromethylvinyl ether copolymer (MFA), and copolymers and derivatives thereof. In some embodiments, outer, expanded layer 12 comprises a polyester, such as poly (ethylene terephthalate) (PET) or a derivative or copolymer thereof. Additional polymers for outer, expanded layer 12 are polyethylene, polypropylene, polyether block amide (PEBA), and polyaryletherketones such as PEEK.

In some embodiments, outer, expanded layer 12 is a non-crosslinked outer layer that comprises, consists essentially of, or consists of one or more polymers such as polyamides, polyethers, polyesters, poly (ether-block-amides); or a copolymer, blend, or derivative of any two or more of the foregoing. Exemplary polymers according to the present disclosure include, but are not limited to, a poly (ether-block-amide) (PEBA) (i.e., a block copolymer comprised of a polyamide segment (e.g., polyamide 6 (PA6), or polyamide 11 (PA11), or polyamide 12 (PA12)), a polyether segment (e.g., polyoxymethylene (POM), or polyethylene glycol (PEG), or polypropylene glycol (PPG), or polytetramethylene glycol (PTMG)), and may include a polyester segment in some grades (e.g., poly (ethylene adipate) (PEA))).

In certain specific embodiments, the non-crosslinked outer layer of the heat shrink tubes comprising PEBA are provided. When referring to the composition of a particular PEBA resin grade, the polyamide segment can be referred to as the “hard segment” or “hard phase”, the polyether segment can be referred to as the “soft segment” or “soft phase”, and the polyester segment (if present in low composition in particular grades of PEBA resin) acts as a chain extender. The ratio of the polyamide, polyether, and polyester segments of the PEBA in the heat shrink tube can vary greatly without departing from the present disclosure. Different composition ratios of the polyamide hard segment to polyether soft segment (in addition to a polyester chain extender in some resin grades) influences the physical properties of the supplied resin; and ultimately the final physical properties of the non-crosslinked PEBA heat shrink tubes of the present invention. Different composition ratios allow for non-crosslinked PEBA heat shrink tubes of varying flexibility (i.e., durometer hardness) to be produced.

In various embodiments, the outer layer of the heat shrink tubing disclosed herein are prepared from one or more poly (ether-block-amide) (PEBA) resins. “Resin” as used herein refers to a material consisting essentially of a given type of polymer (e.g., a copolymer) or two or more polymers/copolymers. Resins are typically provided in solid form (e.g., as solid pellets), although they are not limited thereto (with other forms including, but not limited to, powders, pastes, granules, dispersions, solutions, gels, and the like). In some embodiments, the outer layer of the heat shrink tubing disclosed herein may be prepared from a resin comprising, consisting of, or consisting essentially of a PEBA resin in one or more of the forms noted herein. In some cases, a “resin” as used herein may contain one or more additional components as additives and/or one or more additional components can be added thereto (e.g., such as a lubricant, colorant, filler, and the like). In other embodiments, one or more additional components (in granular, powder, or pellet form or in the form of a gel or liquid) can be included with the PEBA resin and extruded therewith. As such, the outer layer of the heat shrink tube ultimately produced can comprise, in some embodiments, one or more such additional component(s).

In certain embodiments, the outer layer of the heat shrink tubes of the present disclosure are prepared using a PEBA resin, and thus in some embodiments, can consist of PEBA, can consist essentially of PEBA, or can comprise PEBA. Typically, PEBA resins can be provided in a variety of different forms, for example, in the forms of solid pellets, powders, granules, dispersions, solutions, gels, and the like. In certain embodiments, the outer PEBA layer of the heat shrink tubes may be prepared using medical extrusion grade PEBA resin pellets. The type of PEBA resin that is utilized in certain embodiments can vary and may include PEBA medical extrusion grade pellets of different compositions (i.e., different durometer hardness), either as a single PEBA copolymer resin grade, as a blend of two or more PEBA copolymer resin grades, or as a blend that includes a PEBA copolymer resin grade. The PEBA resins utilized in certain embodiments may also be blended or compounded with other polymeric components (e.g., such as polytetrafluoroethylene (PTFE)) to tailor the final properties of the resulting non-crosslinked outer layer of the heat shrink tube for a particular application. Exemplary medical extrusion grade PEBA resins suitable for use according to the present disclosure are commercially available as PEBAX® 7433 SA 01 MED, PEBAX® 7233 SA 01 MED, PEBAX® 7033 SA 01 MED, PEBAX® 6333 SA 01 MED, PEBAX® 5533 SA 01 MED, PEBAX® 4533 SA 01 MED, PEBAX® 4033 SA 01 MED, PEBAX® 3533 SA 01 MED, PEBAX® 2533 SA 01 MED, and PEBAX® MV 1074 SA 01 MED manufactured by Arkema, Inc, or VESTAMID® Care ME71, VESTAMID® Care ME62, VESTAMID® Care ME55, VESTAMID® Care ME47, VESTAMID® Care ME40, and VESTAMID® Care ME26 manufactured by Evonik Corporation. However, it is to be understood that the outer layer of heat shrink tubes provided herein are not limited to PEBA resins and may be prepared using one or more of the polymeric resins described herein in addition to PEBA, or instead of PEBA.

In some embodiments, one or more additives can be incorporated within the bulk of the outer layer, and/or applied upon the outer diameter surface. In some such embodiments, the one or more additives can be distributed (e.g., substantially uniformly) throughout the outer layer thickness and length of the tubing. In some embodiments, the one or more additives may include a lubricant, e.g., such as a thermally stable extrusion process lubricant. In certain embodiments, the lubricant may be a pentaerythritol ester, such as GLYCOLUBER from Azelis Americas, LLC, for example. In some embodiments, the one or more additives may include a radiopaque filler (i.e., an inorganic radiocontrast agent) to assist in medical procedures that utilize fluoroscopy for navigation of a medical device within the body. In certain embodiments, the radiopaque filler may be barium sulfate (BaSO₄), bismuth subcarbonate (Bi₂O₂CO₃), bismuth oxychloride (BiOCl), bismuth trioxide (Bi₂O₃), or tungsten (W), for example. In some embodiments, the one or more additives may include a pigment to provide a desired color of the final outer PEBA layer of the heat shrink tube. In some embodiments, other additives such as inert fillers, stabilizers (i.e., radiation stabilizers, antioxidants, etc.), conductive fillers, anti-tack agents and antimicrobials may be included to produce desired functionality of the final outer PEBA layer of the heat shrink tube for specific applications. The amount of additive that can be contained in the final outer PEBA layer of the heat shrink tube is not particularly limited. In some embodiments, for example, the one or more additives (e.g., lubricant, pigment, filler, etc.) may be included in an amount in the range of 0.1% to 80%, about 1% to about 30%, or about 5% to about 20% by weight based on the total weight of the outer PEBA layer of the heat shrink tube. In other embodiments, the outer PEBA layer of the heat shrink tubes may not include any additives therein.

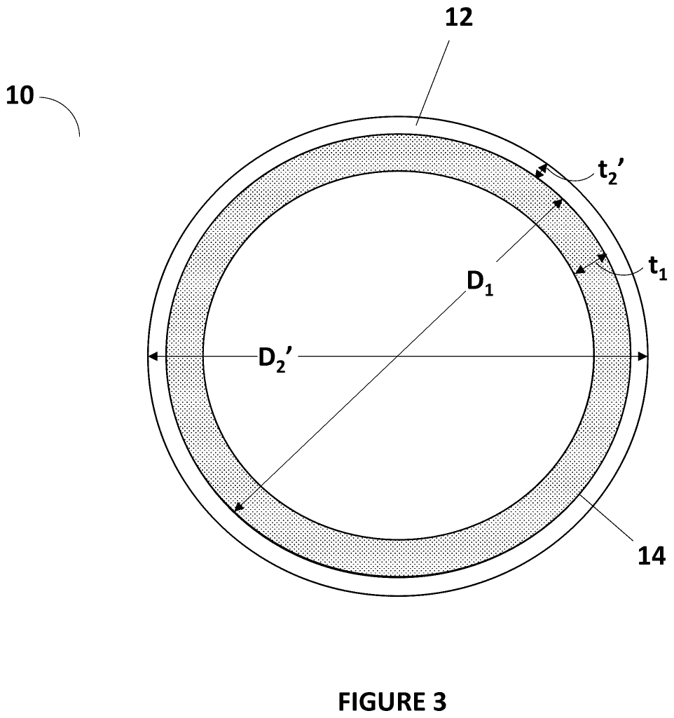

The outer, expanded layer 12 has a wall thickness of t₂′ and an outer diameter of D₂′. As shown in FIG. 3, outer, expanded layer 12 can be prepared, e.g., by providing a tube 11 and expanding it so as to decrease its wall thickness t₂ and to increase its outer diameter D₂. Methods of providing tube 11 are not particularly limited; in some embodiments, tube 11 is an extruded tube. Methods of expanding a tube are known in the art and the present disclosure is not limited to any particular method by which outer, expanded layer 12 is provided. Typically, tube 11 is radially expanded (e.g., by mechanical means) to provide an expanded tubing material that can function as a heat shrink material (i.e., a material which, when heated at or above a particular temperature, returns to its unexpanded form, and consequently “shrinks”). The expansion can be either in-line with production (e.g., extrusion) of tube 11, or offline (i.e., conducted independently of the production of tube 11). The expansion can be done at various temperatures (typically, elevated temperatures) including, but not limited to, temperatures above about 250° C. or above about 300° C. (e.g., about 330-350° C. in certain embodiments). Such embodiments, e.g., as shown in FIG. 3 provide the outer, expanded layer 12 independent of inner layer 14 (and any other layers that may be present within the desired multilayer tubing); however, as referenced herein below, in other embodiments, the expansion of layer 12 is provided by subjecting a composite (e.g., co-extruded) layered structure to expansion (such that inner layer 14 (and any other layers that may be present within the desired multilayered tubing is also subjected to expansion).

All means for radial expansion of tubing are intended to be encompassed by the present invention. In certain embodiments, the tubing is expanded radially by pressurizing the tubing from the inside out, introducing stress into the tube wall. This pressurizing can be conducted by any means capable of providing a differential pressure between the inside and outside of the tubing. Such differential pressure can be created by imposing a pressure above atmospheric pressure in the center of the tube, imposing a pressure below atmospheric pressure on the outside of the tube, or a combination of the two. The stress induced into the wall of the tube causes it to expand outward. The rate of expansion can be controlled so the tube will hold the expanded state and not recover until subjected to a further heat cycle.

The extent to which a tube 11 is expanded depends on the application for which the tubing is intended, and depends, at least in part, on the diameter (D₁) of the inner layer to be combined therewith. The tube 11 is generally expanded at a selected temperature such that D₂′−2×t₂′>D₁. For example, in some embodiments, the tubing is expanded to an outer diameter D₂′ of from about 1.05 times its original (unexpanded) diameter D₂ to about 10 times its original (unexpanded) diameter D₂, such as from about 1.5 times its original (unexpanded diameter D₂ to about 4 times its original (unexpanded) diameter D₂ or about 2 times its original (unexpanded) diameter D₂ to about 4 times its original (unexpanded) diameter D₂. This expansion not only results in an increase in outer diameter D₂ to D₂′, but also results in a decrease in wall thickness (t₂ to t₂′). The values of D₂ and t₂ are not particularly limited; the principles outlined herein are applicable to tubings having a wide range of sizes. In some embodiments, D₂ is about 1 mm to about 30 mm. In some embodiments, D₂′ is about 2 mm to about 50 mm. In some embodiments, t₂′ is about 100 to about 500 μm, although the outer, expanded layer of the disclosed dual layered tubings are certainly not limited to such diameters and wall thicknesses.

In some embodiments, outer, expanded layer 12 is formulated in such a manner as to be easily removed, torn, or peeled away after recovery over the article to be encapsulated, thereby leaving only the inner layer 14 encapsulating the article. Such removability can include, e.g., the outer layer being simply pulled off after recovery. Certain suitable materials and preparation methods for such removable outer, expanded layers are, for example, as described in U.S. Pat. No. 9,440,044 to Roof et al., which is incorporated herein by reference. To promote case of removal, in some embodiments, the material of the outer, expanded layer 12 is selected so as to be largely incompatible with the material of the inner layer 14, e.g., to avoid significant adherence between the two layers (which is likely to occur, e.g., where both layers are fluoropolymeric). One non-limiting example of such incompatibility, which can provide for a removable outer layer is a PEEK outer layer and a FEP inner layer. In some embodiments, the materials are selected such that the outer layer is not easily removable following recovery (e.g., it is intended to remain with the encapsulated component).

Inner layer 14 of the dual layer heat shrink tubing 10 is a tube/layer, including, but not limited to, an extruded tube. In some embodiments, layer 14 is not expanded or oriented (although, during production of the dual layer heat shrink tube, in some embodiments, the layer may be expanded to some extent to provide the dual layer heat shrink tube). The inner layer 14 typically comprises a melt-processable polymer, and in particular, is selected from polymers or blends of polymers that soften and begin to flow before or at the temperature range where the material of the outer layer recovers. Inner polymeric layer 14 can comprise, e.g., a fluoropolymer and, in particular, a thermoplastic melt-processable fluoropolymer. Suitable fluoropolymers are known and include, but are not limited to, fluorinated ethylene propylene (FEP), polyvinylidene fluoride, perfluoroalkoxy alkane (PFA), perfluoro (alkyl vinyl ethers) (PAVE), a tetrafluoroethylene, hexafluoropropylene, vinylidine fluoride terpolymer (THV), polyvinylidene difluoride (PVDF), poly (ethylene-co-tetrafluoroethylene) (ETFE), ethylene chlorotrifluoroethylene (ECTFE), polychlorotrifluoroethylene (PCTFE), ethylene, tetrafluoroethylene, and hexafluoropropylene (EFEP), tetrafluoroethylene and perfluoromethylvinyl ether copolymer (MFA), and copolymers and derivatives thereof. Inner layer 12 can, in some embodiments, comprise polyamides, polyether block amide (PEBA) or polyethylene terephthalate (PET). In some embodiments, inner layer 12 of the dual layer heat shrink tubing 10 can comprise a polyolefin, and, in particular, a thermoplastic melt-processable polyolefin including, but not limited to, polyethylene or polypropylene. In certain embodiments, inner polymeric layer 14 comprises a single type of polymer; in other embodiments, inner polymeric layer 14 comprises two or more polymers (e.g., in the form of a blend).

In some embodiments, inner polymeric layer 14 can further comprise one or more additives, e.g., pigments, dyes, fillers, and the like. Certain non-limiting examples of pigments, dyes, and fillers include, e.g., graphene, carbon nanotubes, clays, organoclays, silica, silicates, zeolites, and functional additives to promote slip, impart color, impart conductivity, or provide antimicrobial characteristics. For example, in one embodiment, inner layer 14 comprises carbon black (e.g., in a relatively low amount, such as about 1% to about 10% by weight).

In some embodiments, the disclosed inner layer (or layers) of the multilayer heat shrink tubes provided herein are non-crosslinked and comprise, consist essentially of, or consist of one or more polymers such as maleated polyolefins (i.e., polyethylenes, polypropylenes, ethylene copolymers), thermoplastic polyurethanes, polyethers, polyesters, ethylene vinyl acetate copolymers, ethylene acrylic acid copolymers, ethylene ethyl acetate copolymers; or a copolymer, blend, or derivative of any two or more of the foregoing. The inner layer(s) of the multilayer heat shrink tubes can comprise, consist essentially of, or consist of one or more polymers that exhibit a melting temperature which is below the melting temperature of the outer layer polymer. Exemplary polymers according to the present disclosure include, but are not limited to, a linear low density polyethylene grafted with maleic anhydride (LLDPE-g-MA).

In various embodiments, the inner layers of the heat shrink tubing disclosed herein are prepared from one or more maleated polyolefin resins. In some embodiments, the inner layers of the heat shrink tubing disclosed herein may be prepared from a resin comprising, consisting of, or consisting essentially of a maleated polyolefin resin in one or more of the forms noted herein. In some cases, a “resin” as used herein may contain one or more additional components as additives and/or one or more additional components can be added thereto (e.g., such as a lubricant, colorant, filler, and the like). In other embodiments, one or more additional components (in granular, powder, or pellet form or in the form of a gel or liquid) can be included with the maleated polyolefin resin and extruded therewith. As such, the inner layers of the heat shrink tube ultimately produced can comprise, in some embodiments, one or more such additional component(s).

In certain embodiments, the inner layer(s) of the heat shrink tubes of the present disclosure are prepared using a maleated polyolefin resin, and thus in some embodiments, can consist of maleated polyolefin, can consist essentially of a maleated polyolefin, or can comprise a maleated polyolefin. Typically, maleated polyolefin resins can be provided in a variety of different forms, for example, in the forms of solid pellets, powders, granules, dispersions, solutions, gels, and the like. In certain embodiments, the inner maleated polyolefin layers of the heat shrink tubes may be prepared using medical extrusion grade maleated polyolefin resin pellets. The type of maleated polyolefin resin that is utilized in certain embodiments can vary and may include maleated polyolefin medical extrusion grade pellets of different compositions (i.e., different maleic anhydride contents), either as a single maleated polyolefin resin grade, as a blend of two or more maleated polyolefin resin grades, or as a blend that includes a maleated polyolefin resin grade. The maleated polyolefin resins utilized in certain embodiments may also be blended or compounded with other polymeric components (e.g., such as polytetrafluoroethylene (PTFE)) to tailor the final properties of the resulting non-crosslinked inner layers of the heat shrink tube for a particular application. Exemplary medical extrusion grade maleated polyolefin resins suitable for use according to the present disclosure are commercially available as OREVAC® 18300M manufactured by Arkema, Inc. or REZILOK RX™ 101 manufactured by Compounding Solutions LLC. However, it is to be understood that the inner layers of the heat shrink tubes provided herein are not limited to maleated polyolefin resins and may be prepared using one or more of the polymeric resins described herein in addition to maleated polyolefins, or instead of maleated polyolefins.

In some embodiments, one or more additives can be incorporated within the bulk of the inner layers, and/or applied upon the inner diameter surface. In some such embodiments, one or more additives can be distributed (e.g., substantially uniformly) throughout the inner layer thickness and length of the tubing. In some embodiments, the one or more additives may include a radiopaque filler (i.e., an inorganic radiocontrast agent) to assist in medical procedures that utilize fluoroscopy for navigation of a medical device within the body. In certain embodiments, the radiopaque filler may be barium sulfate (BaSO₄), bismuth subcarbonate (Bi₂O₂CO₃), bismuth oxychloride (BiOCl), bismuth trioxide (Bi₂O₃), or tungsten (W), for example. In some embodiments, the one or more additives may include a pigment to provide a desired color of the final inner maleated polyolefin layers of the heat shrink tube. In some embodiments, other additives such as inert fillers, stabilizers (i.e., radiation stabilizers, antioxidants, etc.), conductive fillers, anti-tack agents and antimicrobials may be included to produce desired functionality of the final inner maleated polyolefin layers of the heat shrink tube for specific applications. The amount of additive that can be contained in the final inner maleated polyolefin layers of the heat shrink tube is not particularly limited. In some embodiments, for example, the one or more additives (e.g., lubricant, pigment, filler, etc.) may be included in an amount in the range of about 0.1% to about 80%, about 1% to about 30%, or about 5% to about 20% by weight based on the total weight of the inner maleated polyolefin layer of the heat shrink tube. In other embodiments, the inner maleated polyolefin layers of the heat shrink tubes may not include any additives therein.

The size of the inner polymeric layer 14 can vary, although it is generally sufficiently small enough in outer diameter D₁ so as to fit within the inner diameter of expanded tube/layer 14. Similarly, the wall thickness t₁ of inner polymeric layer 14 is not particularly limited. In some embodiments, t₁ is about 25 μm to about 900 μm, although the dual layer tubings provided herein are not limited to such thicknesses. In some embodiments, t₁ is greater than t₂′ (i.e., the thickness of outer, expanded layer 12); in other embodiments, t₁ is less than t₂.′

According to the present disclosure, outer, expanded layer 12 and inner layer 14 are selected and combined in a particular manner designed to provide a suitable dual layer heat shrink tube. The properties of the individual resins selected for each layer, as well as the manner of expansion of the outer polymeric layer 12 (e.g., the rate, extent, and temperature of expansion) affect the overall performance of the resulting composite heat shrink tubing 10.

In some embodiments, the inventors have evaluated certain interactions, e.g., taking into consideration the melt properties of inner layer 14, which advantageously is designed to melt/flow and the heat shrink properties of outer, expanded layer 12, which advantageously is designed to effectively encapsulate any underlying component(s). Mismatches between the melting/flowing of the inner layer and the recovery temperature and force of the outer, expanded layer can cause issues during encapsulation of a component (e.g., including, but not limited to, the presence of voids in the recovered/heat shrunk tube, unacceptable elongation of the tube, and overflow of the inner layer polymer outside the ends of the recovered tube). Certain principles of the present disclosure serve to address certain such difficulties in balancing the various properties of a dual layer heat shrink tube and obtain good encapsulation.

For example, such difficulties include, but are not limited to, the fact that the melting range of the inner layer polymer can be very large (e.g., spanning 40-50° C. for some polymers), thus making it difficult to determine the point at which the bulk of the melting polymer mass can flow to effectively encapsulate a part when a normal force is applied during heating. Moreover, the force applied by the shrinking tube as well as the melt viscosity are themselves functions of temperature. The situation can become further complicated if the method of heating the dual shrink tube does not operate at a steady temperature in the field application (i.e. through use of a heat gun). In addition, the viscosity of the inner layer polymer will vary with temperature and polymer type and grade, and the recovery force of the outer layer will also be a function of temperature as well as of wall thickness and expansion process parameters. Furthermore, the recovery process can itself be non-isothermal.

According to the present disclosure, dual layer heat shrink tubes can be provided that result from an evaluation process that considers the complex interactions of both layers in a non-isothermal environment and takes into consideration the concerns referenced above. More specifically, the disclosed method accounts for melt/flow and recovery characteristics simultaneously by evaluating the dynamic mechanical response of the entire composite structure over a wide range of temperatures. Thus, multilayer (e.g., dual layer) heat shrink tubes can be designed based on the net effect of the temperature-dependent deformation and flow of the combined structure. The inventors have recognized a method by which the interactions of complex phenomena in a dual layer heat shrink tubing can be understood so that a melting inner layer can be effectively deformed around a component by the recovering outer layer (during heat shrink deployment). Dual layer heat shrink tubings manufactured using the disclosed method can be thus optimized for recovered encapsulation quality. This process allows for efficient design of dual layer heat shrink tubings by combining an outer recoverable tube with an inner melting tube in a rapid DMA test to determine suitable/optimal combinations.

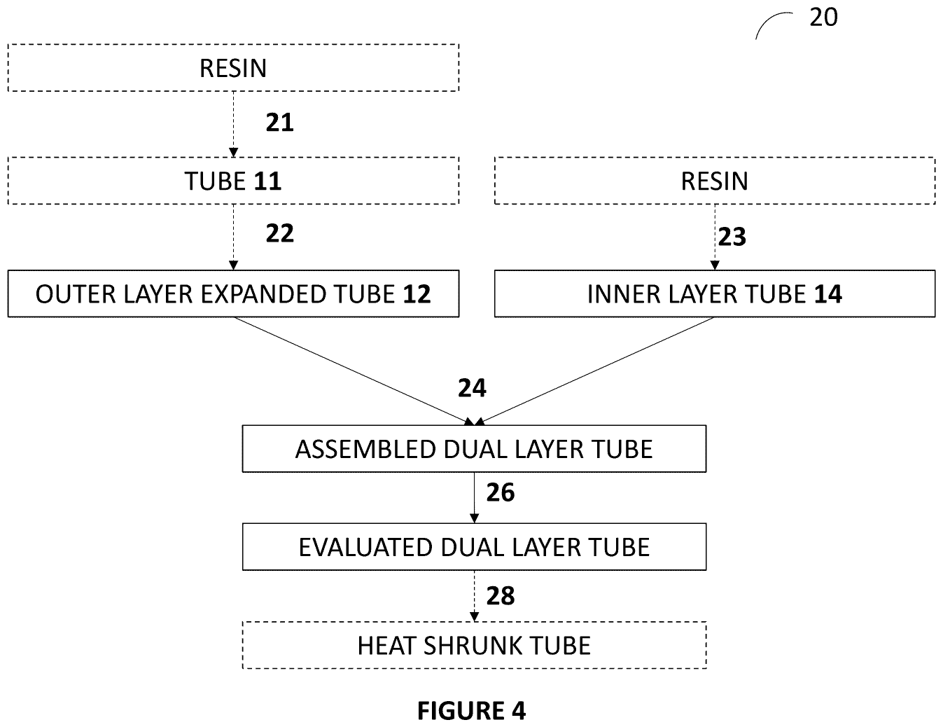

A non-limiting method for production of dual layer heat shrink tubes is shown in FIG. 4. As referenced above, the method generally involves combining an inner layer tube with an outer, expanded tube to give an assembled dual layer tube. Steps 21 (preparation of a tube, e.g., extrusion of a resin into a tube), 22 (expansion of the tube) and 23 (preparation of a tube, e.g., extrusion of a resin into a tube) are optional in the context of the disclosed process (i.e., one or both of inner layer tube and an outer layer expanded tube can be prepared via steps 21, 22, and/or 23) or these tubes can be provided and subjected to assembly step 24.

Assembly step 24 involves combining the inner and outer layers, both in tube form. These layers can be combined in conventional manners. For example, in some embodiments, the inner layer 14 is inserted into the outer, expanded layer 12 to form the dual layer heat shrink tube. In some embodiments, the inner layer 14 is expanded to some extent within and into outer, expanded layer 12, e.g., via the application of heat and/or pressure. In some embodiments, the outer, expanded layer 12 is partially recovered over the inner layer 14, e.g., by heating the outer layer.

Although, in some embodiments, individual layers are provided and combined to form a multilayered tubing as provided herein, the disclosure is not limited thereto. For example, in some embodiments, desired resins for the layers are provided and extruded together (and expanded together, such that both layers are, in some embodiments, expanded), as described herein below. Generally, the desired resin or resins, such as the PEBA and maleated polyolefin resins as described herein, are converted into a multilayer tubular form via extrusion and then mechanically expanded (such that, in the multilayer heat shrink tubing, both layer 12 and layer 14 are in expanded form). The means by which these steps are conducted can vary, as will be described herein.

A multilayer tube may be formed through the multilayer extrusion process. Multilayer extrusion generally comprises placing the desired resin or resins into an extruder (e.g., a single screw melt extruder) designated for a given layer or layers. Within each extruder, the resin or resins are heated, compressed, and conveyed out of the extruder and through a multilayer extrusion head (also referred to herein as a “multilayer head” or simply “head”) containing an annular die set, creating a multilayer tube. The annular die set (also referred to herein as “tooling”) consists of a circular extrusion die and a mandrel which forms the polymer melt into a tubular form as it exits the multilayer extrusion head. Within the multilayer head the polymer melts from each of the extruders are brought together with minimal mixing to allow the formation of distinct layers within the tube.

Multilayer tubes of various diameters, wall thicknesses, and lengths can be produced using the forming methods described herein. The final dimensions of the extruded multilayer tubular form can be adjusted and optimized through proper tooling selection along with other parameters in the extrusion step such as temperature, pressure, and screw rotation speed. The head is connected to a number of extruders, each of which is generally comprised of a hopper, barrel, screw, and breaker plate. The screw of each extruder is generally comprised of several sections (e.g., the feed, compression, and metering zones) that can be optimized to provide an effective and consistent extrusion process. Generally, there are multiple temperature-controlled zones throughout each extruder, each of which can be adjusted and optimized to produce tubular forms of desired dimension and quality. In some embodiments, multilayer tubing having a relatively uniform total wall thickness (i.e., high percent concentricity) is provided.

Appropriate sizing of the tooling to be used during the extrusion process is generally determined by the specified finished multilayer tubular dimensions, individual extruder specifications, the desired draw down ratio (DDR), and the draw ratio balance (DRB). The DDR and DRB are unitless quantities used by those skilled in the art to describe the relationship between the dimensions of the polymer melt as it exits the tooling to the dimensions of the final multilayer tubular form (i.e., the final extruded tube dimensions). Draw down ratio (DDR) is defined as the ratio of the cross-sectional area of the polymer melt as it exits the tooling to the cross-sectional area of the final tubular form. The molten tubular form must be “drawn down” (i.e., reduced in diameter and cross-sectional area via stretching) after exiting the tooling and before quenching (i.e., rapidly cooling in air or a chilled fluid bath) to obtain the desired final tubular dimensions. Generally, utilizing tooling that provides a high DDR with respect to the dimensions of the final tubular form enables faster line speeds (i.e., a faster production rate). It is also important to note that a tube produced with a higher DDR has a greater degree of longitudinal orientation of the polymer chains than a tube produced using a lower DDR. It is well known to those skilled in the art that imparting a particular degree of orientation on a polymeric material can influence the mechanical and physical properties of the final product. These properties (e.g., such as mechanical properties) can be optimized for a particular application (i.e., input for a secondary expansion process) by varying the degree of orientation. Draw ratio balance (DRB) is defined as the diameter ratio of the extrusion die and mandrel divided by the diameter ratio of the final tubular form. Generally, DRB characterizes the relationship between the annular shape of the polymer melt as it exits the die and the annular shape of the final tubular form. It is well known by those skilled in the art that careful tooling selection is required to achieve a stable tubular extrusion process. The dimensional relationship of the tooling used to produce a tube of a given size influences the line speed of the extrusion process (i.e., the production rate) based on the DDR. However, this dimensional relationship must be balanced because it also directly influences the overall stability of the extrusion process through the DRB. Tooling selection is an important aspect of the extrusion process to produce a tubular form of specified dimension that also has desirable mechanical characteristics.

The extruded multilayer tubular form is then typically radially expanded (e.g., by mechanical means) to provide an expanded multilayer tube, i.e., a multilayer heat shrink tube (i.e., a tubing which decreases in diameter when heated). The expansion of the input multilayer tubing (i.e., the initial extruded tubular form) can be conducted in-line with extrusion or off-line (i.e., conducted independently of and/or secondary to the extrusion process). All means for radial expansion of tubing are intended to be encompassed by the present invention. Generally, during the expansion process, the multilayer tubing is expanded radially by pressurizing the inside of the tubing, introducing stress into the tube wall. This pressurizing can be conducted by any means capable of providing a differential pressure between the inside and outside of the tubing. Such differential pressure can be created by imposing a pressure above atmospheric pressure on the inside of the tube, imposing a pressure below atmospheric pressure on the outside of the tube, or a combination of the two. The stress induced into the wall of the tube causes it to expand radially, i.e., increase in diameter. The rate of expansion can be controlled so the tube will hold the expanded state and does not recover until subjected to a further heat cycle. The extent to which a tube is expanded depends on the application for which the final heat shrink tubing is intended. The rate and extent to which a tube is expanded depends on the temperature at which the expansion process is conducted. It has been found that the expansion chamber temperature must be carefully controlled to optimize the rate and extent of expansion of the tube. In some embodiments, the tubing is expanded to an inner diameter from about 1.05 times its original (unexpanded) inner diameter to about 10 times its original (unexpanded) inner diameter.

In certain embodiments, multilayer PEBA heat shrink tubes prepared according to the present disclosure may be radially expanded using the processes described, for example, in U.S. Pat. No. 9,296,165 to Henson, which is incorporated by reference herein in its entirety. For example, the Henson patent describes a process for the production of thermoplastic polymeric heat shrink tubing using a first fluid in the interior of a tube to expand it and a second fluid exterior to the tube to constrain the expansion within an expansion chamber. In other embodiments, for example, the tubing may be expanded by adjusting the flow rate of the air external to the tube, the chamber temperature, the air pressure within the tube, and the rate at which the tube moves through the expansion chamber. In certain embodiments, the heat shrink tubes of the present disclosure are expanded at elevated temperature through a die using any number of methods known to the art, and subsequently cooled at the die exit. Cooling can be accomplished using fluids such as water, oil, or air. The processing parameters that can be adjusted include, but are not limited to: die type, die diameter and length, die temperature, fluid pressure inside the tube, fluid pressure outside the tube, cooling method, cooling medium type and temperature, expansion rate, tube materials, tube ID, tube OD, and tube wall thickness.

It is noted that, although certain heat shrink PEBA tubes are known, these tubes comprise predominantly crosslinked PEBA, which provides for the heat shrink capabilities of the tubes. Generally, the PEBA heat shrink tubes provided herein are expanded under carefully controlled conditions of internal air pressure, temperature, and throughput through an expansion die prior to rapidly cooling the tube to lock in the entropically unfavorable expanded state. Examples of heat shrinkable crosslinked PEBA tubing are provided, for example, by Cobalt Polymers, TE Connectivity, and in the disclosures of U.S. Pat. No. 7,306,585 to Ross and U.S. Pat. App. Pub. No. 2008/0317991 to Pieslak et al. Crosslinking through chemical means or by irradiation has long been used in the production of heat shrink tubes and films in order to obtain a greater degree of clastic recovery of the expanded part upon heating (i.e., increase attainable recovery ratio and/or recovery force upon heating). Crosslinking a polymer article such as a tube or film effectively increases the molecular weight of the polymer in addition to improving its elastic response to an imposed deformation. This effective increase in molecular weight also results in a marked increase in the viscosity of the polymer (due to the increased probability of interchain entanglements), which hinders the ability of the material to flow into and fill voids (i.e., open spaces or interstices in the pattern of an underlying reinforcing component). The increased viscosity due to crosslinking also reduces the ability of the polymer to conform tightly to and bond with an underlying substrate during recovery. The increased recovery ratio attainable with crosslinked materials can also lead to unwanted deformation or damage to a sensitive underlying substrate during recovery.

In some embodiments, upon assembly, a given dual layer heat shrink tube is subjected to evaluation 26 to determine whether the tube will serve as an effective heat shrink tube (with an effective heat shrink tube defined for purposes herein, e.g., as a heat shrunk material exhibiting few voids, little overflow, and change in recovered length less than about 20% the original length). The unique evaluation conducted at this stage is based, at least in part, on evaluating the area under the tan δ curve of the dual layer heat shrink tube in a DMA instrument undergoing a heating ramp. It has been discovered that for some embodiments, tan δ of the composite tube is influenced by both the softening and melting of the inner polymeric layer 14, and the recovery of the outer layer 12. In an increasing temperature ramp, tan δ grows rapidly as the inner layer softens and melts. Thus, the larger the integral of tan δ with temperature, the greater the propensity of the inner tube polymer to flow freely as it is heated. Eventually when the expansion temperature is reached, the recovery forces exerted by the outer layer 12 will cause tan δ of the composite structure to decrease as the polymer flow becomes increasingly constrained. The storage modulus, E′, of the outer tube decreases with temperature until the expansion temperature is reached. At this point, a noticeable kink is seen in E′ followed by an increase as the outer layer recovers. A kink in E′-T curve has been attributed to entropic elasticity such as would be seen in the recovery of a previously expanded sheet. See L. Andena et al., Polym. Eng. Sci, 44, 2004, 1368-1378, which is incorporated herein by reference. The E′ curve decreases with temperature once more after all stresses have been relieved. It has now been found that the area under the tan δ curve calculated from the onset of melting of the inner tube through the onset of melting of the outer tube can be used to describe these competing phenomena. If the value of the area under the tan δ curve is below about 1° C., it had been found that the mobility of the polymer within the inner tube is insufficient to properly encapsulate the underlying component. If the value of this area is above about 12° C., it has been found the polymer within the inner tube flows freely outside the confines of the outer tube and does not properly encapsulate the underlying component. As such, the targeted value according to the present disclosure is 1° C. to 12° C., e.g., 2° C. to 12° C., 3° C. to 12° C., 4° C. to 12° C., or 5° C. to 12° C.

The evaluation 26 comprises providing the assembled dual layer tube to be evaluated, evaluating the area under a tan δ curve when the assembled dual layer tube is cut into a ring, slit into a rectangle and gripped by tension grips holding the cut ends of the rectangle within a DMA, and subjected to a temperature sweep of 3° C./min at a frequency of 1 Hz from the onset of a melting endotherm of the inner layer (as determined by DSC) up to the onset of a melting endotherm of the outer layer (as determined by DSC). It is determined that the combination of inner and outer layer provide a suitable combination of inner and outer layers if: a) the area under the tan δ curve is less than about 12° C. and greater than about 1° C. By ensuring that a given combination of inner and outer layers exhibits such properties, the inventors have found it can be reasonably predicted that a dual layer heat shrink tube has been produced, which will exhibit acceptable heat shrink/encapsulation properties upon recovery, e.g., few voids, little overflow) with a change in recovered length less than about 20% of the original length. This novel procedure allows for rapid design of dual layer heat shrink tubing by combining an outer, recoverable layer with an inner, melting layer in the DMA and evaluating the results, without the need to produce production quantities of such tubings to test.

In some embodiments, heat shrink tubes with unique properties and unique combinations of properties are provided. As generally noted above, a heat shrink tube is a shrinkable tubing prepared via expansion of a polymeric (“input”) tubing (e.g., an extruded tubing) to give the heat shrink tubing (also referred to herein as an “expanded” form). Upon heating, the heat shrink tubing “shrinks” to a size that is equivalent to (or close to) its original/input size, commonly referred to as its “recovered” size. A heat shrink tubing can be defined, e.g., by measurable properties such as its inner diameter (“ID”) either after expansion (also referred to herein as “expanded inner diameter” (IDₑ)) or after recovery (also referred to herein as “recovered inner diameter” (IDᵣ)), its length (L), its change in length upon recovery (i.e., its percent change in length upon recovery, ΔL), its average wall thickness, its ratio of individual average layer thicknesses (also referred to herein as “layer ratio”), its wall thickness concentricity (also referred to herein as percent concentricity or simply as concentricity), its expansion ratio (ER), its recovery ratio (RR), and its percent change in inner diameter upon recovery (ΔID). Such properties can be defined using the following equations:

$$\begin{matrix}Expansion ratio = ER = \frac{{ID}_{e}}{{ID}_{o}} \\ Recovery ratio = RR = \frac{{ID}_{e}}{{ID}_{r}} \\ \% Change in Length = Δ L = \frac{{L}_{r} - {L}_{e}}{{L}_{e}} ( 100 ) \\ \% Change in Inner Diameter = Δ ID = \frac{{ID}_{e} - {ID}_{r}}{{ID}_{e}} ( 100 ) \\ \% Concentricity = \frac{{wt}_{min}}{{wt}_{max}} ( 100 ) \\ Layer Ratio = \% {wt}_{out} : \% {wt}_{in} = \frac{{wt}_{out}}{{wt}_{total}} ( 100 ) : \frac{{wt}_{in}}{{wt}_{total}} ( 100 )\end{matrix}$$

In these equations, Lₑ and Lᵣ are the length of the heat shrink tubing (in expanded form) and the length of the “recovered” (i.e., heat-shrunk) tubing, respectively. IDₒ refers to the original internal diameter (ID) of the input tube (i.e., the tube before it is expanded and then subsequently “shrunk”); IDₑ refers to the internal diameter (ID) of the expanded heat shrink tubing; and IDᵣ refers to the internal diameter (ID) of the recovered (heat shrunk) tube. Values needed for determination of percent concentricity are the minimum wall thickness and the maximum wall thickness of the tubular walls, defined as wtₘᵢₙ and wtₘₐₓ, respectively. wtₒᵤₜ refers to the average thickness of the outer layer; wtᵢₙ refers to the average thickness of the inner layer(s); and wtₜₒₜₐₗ refers to the total average wall thickness. RR, ΔL, and ΔID can be evaluated under any recovery conditions (i.e., time, temperature, and method of heat application), though the time and temperature at which an expanded tube is recovered must be specified as this can influence the observed extent of recovery (i.e., an expanded tube that is exposed to a lower temperature and/or for a shorter time may not recover to its full capability). Percent concentricity can be evaluated in the expanded or recovered state. Concentricity is a measure of wall thickness uniformity, and the concentricity value can influence performance in certain applications in both states. As used herein, the above parameters were calculated as follows.

The percent change in length (ΔL), also referred to herein as longitudinal change, is determined in the following manner. Prior to placing the heat shrink tubing into the oven for unrestricted recovery, the expanded tubing is cut to a length of 2.5 inches using a verified ruler. The 2.5-inch specimen length is carefully cut from the heat shrink tubing to ensure there are no burs or other deformities present, and that they are perpendicular to the longitudinal axis of the tubing. After the unrestricted recovery process at a specified temperature, the tubing length is re-measured using a verified ruler to the nearest 1/32nd of an inch to determine the amount of shrinkage or growth that has occurred during the process. For example, the expanded length is subtracted from the recovered length and divided by the expanded length, then this quantity is multiplied by 100 to give the overall percent change in length (ΔL) resulting from recovery. Typically, ΔL is measured to be in the range of about +/−10% (i.e., the length changes by less than about 10% upon recovery).

The recovery ratio (RR), percent change in inner diameter (ΔID), and percent concentricity is determined in the following manner. Three 2.5-inch-long specimens are cut from the expanded tubing and their expanded ID and wall thickness is measured using verified measurement tools. Multiple wall thickness measurements must be taken to accurately determine the percent concentricity (i.e., the wall thickness uniformity) of the tubular walls. The minimum wall thickness measurement taken on the expanded tube is divided by the maximum wall thickness measurement taken on the expanded tube, and then multiplied by 100 to give the percent concentricity of the expanded tube. The specimens are then placed into an oven set at a specified temperature for approximately 10 minutes. After exposing each heat shrink tubing specimen to a specified recovery temperature for 10 minutes, it is removed from the oven and allowed to cool to ambient temperature. This subjects the expanded heat shrink tubing to an unrestricted recovery process. After cooling to ambient temperature, the recovered ID and wall thickness is measured using verified measurement tools. The expanded tubing ID is divided by the recovered tubing ID to calculate the recovery ratio (RR) of the heat shrink tube under the specified recovery conditions (i.e., recovery temperature and time). Subsequently, the percent inner diameter change of the heat shrink tubing is calculated by subtracting the recovered tubing ID from the expanded tubing ID and dividing by the expanded tubing ID, then multiplying this quantity by 100 to give the overall percent change in inner diameter (ΔID). The minimum wall thickness measurement taken on the recovered tube is divided by the maximum wall thickness measurement taken on the recovered tube, and then multiplied by 100 to give the percent concentricity of the recovered tube.

Advantageously, as referenced herein, in some embodiments, multilayer heat shrink tubes comprising an outer layer of PEBA and an inner layer of maleated polyolefin are provided, wherein a majority (e.g., the entirety) of the PEBA within the outer layer and the maleated polyolefin within the inner layer of the tube are not cross-linked. In some embodiments, the tubes provided herein comprise no cross-linked polymer, comprise less than 2% by weight of a crosslinked polymer, less than 1% by weight of a cross-linked polymer, or less than 0.5% by weight of a cross-linked polymer. The disclosed multilayer heat shrink tubes can exhibit high recovery ratios; in some embodiments, the disclosed multilayer heat shrink tubes can have recovery ratios (RRs) of greater than about 1.05:1, greater than about 1.10:1, greater than about 1.2:1, greater than about 1.3:1, greater than about 1.4:1, greater than about 1.5:1, or greater than about 1.6:1, e.g., about 1.05:1 to about 2:1. In some embodiments, the multilayer heat shrink tubes can be described based on the reducibility in ID (upon recovery). Examples of such values include, but are not limited to, tubes reducible in ID by at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, or at least about 30%, such as about 5% to about 40%, including, e.g., about 4.8%, about 9.1%, about 16.7%, about 23.1%, about 28.6%, about 33.3%, or about 37.5%.

In some embodiments, the disclosed multilayer heat shrink tubes can be described based upon their durometer hardness measurements of the outer layer. For example, in one specific embodiment, a durometer hardness measurement of a heat shrink tubing as provided herein according to ASTM D2240 conducted on a flat specimen fabricated by melt pressing the heat shrink tubing (in expanded form) is about 20 to about 80 shore D.

The multi-layer heat shrink tubings provided herein can find application in a range of fields. For example, they may be used in various medical contexts, as well as in environmental, medical, chemical, electrical, optical, electronic, aerospace, automotive, and telecommunications fields. As such, the disclosure includes components (e.g., environmental, medical, chemical, or electrical, optical, electronic, aerospace, automotive, or telecommunication components) encapsulated within a tubing, e.g., prepared by recovering a dual layer heat shrink tubing around such components). Similarly, methods for encapsulating components effectively (e.g., meeting the criteria mentioned herein) are encompassed by the disclosure. These methods can result in components encapsulated by the two layers provided herein (the recovered outer layer and the melted inner layer) and, in some embodiments, where the recovered outer layer is removable, the final encapsulated component can comprise only a coating of the inner layer material.

In some embodiments, they are advantageously used in catheter assembly processes, e.g., as a replacement for conventional (e.g., FEP) heat shrink tubes used as manufacturing aids to compress an underlying catheter pre-assembly during heating/reflow. Such processes can avoid the need for disposable components, e.g., the FEP heat shrink tubes typically required to produce the catheter assembly. According to the present disclosure, the provided multilayer heat shrink tubes (e.g., PEBA heat shrink tubes with an adhesive inner layer) can function both as a catheter jacket and as a processing aid/heat shrink to provide sufficient compression during heating/reflow.

As such, the disclosed multilayer heat shrink (i.e., expanded) tube enables a catheter shaft to be manufactured by heating the multilayer heat shrink tube as an outer sheath tube (“jacket”) of a catheter shaft pre-assembly, inducing dimensional recovery of the multilayer heat shrink tube and allowing for reflow of the inner layer(s) through a reinforcing component (where present) and bonding to the underlying liner without the use of a fusing sleeve (e.g., FEP). Since the polymeric material of the disclosed multilayer heat shrink tube is not crosslinked, its viscosity is such that the polymer can easily flow through and encapsulate a reinforcing component (where present) and adhere to the outer surface of the inner liner when heated under an appropriate recovery profile. The selection of an appropriate heating temperature and time (also referred to herein as “heating profile” or “recovery profile”) for a particular non-crosslinked multilayer heat shrink outer jacket tube can vary substantially depending on the underlying catheter assembly components and the composition of the non-crosslinked multilayer heat shrink tubing. In this way, expansion conditions, recovery ratio, and the recovery profile can be tailored to provide a non-crosslinked PEBA heat shrink tube with an adhesive inner layer capable of forming an outer sheath for various different types of catheter structures.

After production of the desired construct with the disclosed multilayer heat shrink tube associated therewith and during or after recovery of the multilayer heat shrink tube, if desired, the outer surface of the construct (comprising the multilayer heat shrink material) can optionally be further modified or smoothed through a secondary process. For example, in some embodiments, the construct is passed through a heated metallic die or a heated polymer-coated die during or after recovery of the multilayer heat shrink material, producing a catheter assembly comprising a modified (e.g., smoothed) outer surface. The heated die can be coated with polytetrafluoroethylene (PTFE), for example. In some embodiments, the construct is passed through a heated or polymer-coated die during or after recovery of the multilayer heat shrink material to provide a textured or engineered outer surface to modify or enhance sliding properties of the finished construct.

The use of the disclosed multilayer heat shrink tubing as an outer jacket for catheter shafts removes the need for manufacturing aids such as FEP heat shrink tubes commonly used in the production of catheter shafts. This is advantageous in many respects. Firstly, an expensive component is removed from the manufacturing process. Secondly, process scrap is reduced since the FEP heat shrink tubing must be removed and discarded after the reflow process is completed. Thirdly, the heat transfer of the recovery process is improved greatly by removing, in effect, an insulating layer (i.e., the FEP heat shrink tubing), which will improve cycle times. Finally, the potential for damaging the completed catheter shaft during manufacturing is greatly reduced if there is no need to nick, skive, or cut away an FEP heat shrink tube in order to remove it.

### EXAMPLES

Aspects of the present invention are more fully illustrated by the following examples, which are set forth to illustrate certain aspects of the present invention and are not to be construed as limiting thereof.

A TA Instruments DSC Q2000 was used to study the thermal behavior of both the inner and outer tubes of the composite sample in order to determine the onset of the melting region for both inner and outer tubes. The sample was first equilibrated at −60° C. and heated at a constant rate of 10° C./min to 400° C. The onset of the melting region was then determined using TA Instruments Universal Analysis 2000 v4.5A software.

A TA instruments Q800 DMA equipped with the film-tension fixture operating in tension mode was used to determine the thermo-mechanical properties of the composite tube, with the main property of interest being tan delta (8). The composite tube was cut perpendicular to its length forming an annulus that was subsequently slit open into a rectangular test specimen. Both layers of the rectangular test specimen were placed in the tension grips with the slit circumference of the tube thus being parallel to the plane of the grips. A temperature ramp was performed at a constant rate of 3° C./min and the test frequency was 1 Hz. Upon completion of the temperature scan, the DMA data was imported into TA instruments TRIOS software v4.3. Raw data files were exported to MS Excel 2016. The Excel data files were then imported into OriginLab's OriginPro 2020 v.9.7 data analysis and graphing software and tan delta vs. temperature plotted. Using OrginLab's integration gadget, the area of the tan delta with respect to temperature was calculated with the lowermost temperature limit being the onset temperature of the melting endotherm of the inner tube determined by DSC and the uppermost temperature limit being the onset temperature of the melting endotherm of the outer tube determined by DSC with the region of interest (ROI) being between these two limits. For all calculations, the baseline was set at y=0 (i.e., tan δ=0).

The encapsulation quality was evaluated as follows. The composite tube having a length of about 15 cm was placed over a mandrel that was equal to 60%±10% of the inner diameter of the composite tube. Using a forced air oven, recovery was performed at a temperature higher than the initial expansion temperature. At 10 minutes the encapsulated mandrel was removed from the oven and set aside to cool to ambient. Once cool, the outer layer of the recovered composite tube was measured to determine percent longitudinal change. An increase in length exceeding 15% of the initial length, or a reduction in length exceeding 2% of the initial length was indicative of a faulty encapsulation. A visual inspection was then performed to ensure the absence of large voids, i.e. exceeding 2 mm in diameter. If the overflow of the molten polymer from each end of the recovered outer tube was greater than 5 mm in width, the encapsulation was deemed to be faulty.

### Comparative Example 1

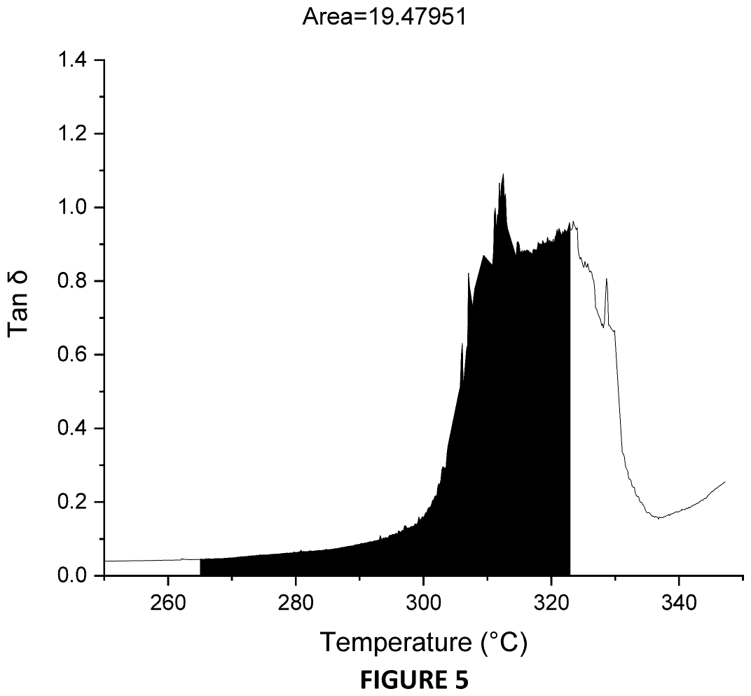

A dual layer heat shrink tube with initial outer diameter of 3.59 mm was manufactured with an outer layer of PTFE (104 μm wall thickness) expanded between 330° C. and 350° C. at a ratio of 2:1 and an inner layer of PFA (315 μm wall thickness). The onset of melting from the DSC endotherm was determined to be 265° C. for the PFA. The onset of melting from the DSC endotherm was determined to be 323° C. for the PTFE. Recovery was carried out in a hot air circulating oven at 350° C. for 5 minutes. A DMA temperature sweep was carried out at 1 Hz on a rectangular sample cut from the circumference of the original composite tube and slit open. The area under the tan δ curve from 265° C. to 323° C. was determined to be 19.48° C. The E′ curve showed a positive slope around the recovery temperature. Upon recovery/heat shrink at 350° C. over a mandrel having a diameter of 1.74 mm, the encapsulation of the mandrel was found to have voids with a change in length of-4.6%, but with significant overflow of the PFA layer. FIG. 5 shows a DMA temperature sweep of the dual layer heat shrink tube, showing calculation of tan δ area from 265° C. to 323° C.

### Example 1

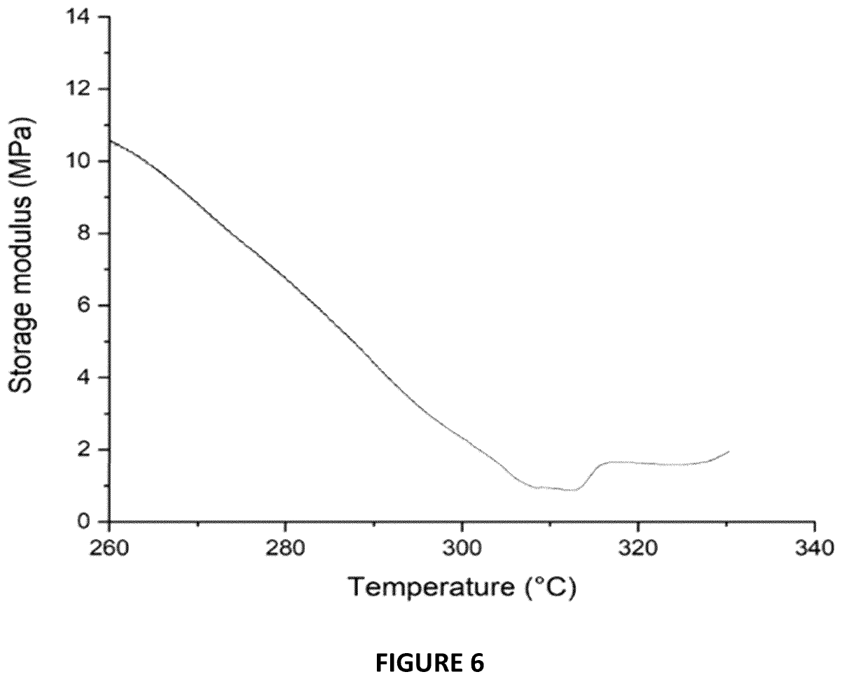

A dual layer heat shrink tube with initial outer diameter of 3.52 mm was manufactured with an outer layer of PTFE (with a wall thickness of 107 μm) expanded between 33° and 350° C. at a ratio of 2:1 and an inner layer of PFA (with a wall thickness of 219 μm). The onset of melting from the DSC endotherm was determined to be 265° C. for the PFA. The onset of melting from the DSC endotherm was determined to be 323° C. for the PTFE. The tube was recovered/shrunk over a mandrel of 1.48 mm at 350° C. for 10 minutes. A DMA temperature sweep was carried out at 1 Hz on a rectangular sample cut from the circumference of the original composite tube and slit open. The results are shown in Table 1. FIG. 6 shows the change in modulus for the dual layer heat shrink tube as a function of temperature.

### Example 2

A dual layer heat shrink tube with initial outer diameter of 8.37 mm was manufactured with an outer layer of PTFE (with a wall thickness of 162 μm) expanded between 33° and 350° C. at a ratio of 2:1 and an inner layer of PFA (with a wall thickness of 315 μm). The onset of melting from the DSC endotherm was determined to be 265° C. for the PFA. The onset of melting from the DSC endotherm was determined to be 323° C. for the PTFE. The tube was recovered/heat shrunk over a mandrel of 3.83 mm at 350° C. for 10 minutes. A DMA temperature sweep was carried out at 1 Hz on a rectangular sample cut from the circumference of the original composite tube and slit open. The results are shown in Table 1.

### Comparative Example 2

A dual layer heat shrink tube with an initial outer diameter of 5.85 mm was manufactured with an outer tube of unexpanded PTFE (365 μm wall thickness) and an inner tube of PVDF (445 μm wall thickness). The onset of melting from the DSC endotherm was determined to be 160° C. for the PVDF. The onset of melting from the DSC endotherm was determined to be 323° C. for the PTFE. The dual layer heat shrink tube was recovered over a mandrel of 4.37 mm at 350° C. for 10 minutes. A DMA temperature sweep was carried out at 1 Hz on a rectangular sample cut from the circumference of the original composite tube and slit open. The results are shown in Table 1.

### Example 3

A dual layer heat shrink tube with initial outer diameter of 4.22 mm was manufactured with an outer layer of PTFE (with a wall thickness of 260 μm) expanded between 33° and 350° C. at a ratio of 2:1 and an inner layer of PFA (with a wall thickness of 315 μm). The onset of melting from the DSC endotherm was determined to be 265° C. for the PFA. The onset of melting from the DSC endotherm was determined to be 323° C. for the PTFE. The tube was recovered/heat shrunk over a mandrel of 1.91 mm at 350° C. for 10 minutes. A DMA temperature sweep was carried out at 1 Hz on a rectangular sample cut from the circumference of the original composite tube and slit open. The results are shown in Table 1.

### Comparative Example 3

A dual layer heat shrink tube with initial outer diameter of 14.06 mm was manufactured with an outer layer of PTFE (with a wall thickness of 251 μm) expanded between 33° and 350° C. at a ratio of 2:1 and an inner layer of FEP (with a wall thickness of 750 μm). The onset of melting from the DSC endotherm was determined to be 235° C. for the FEP. The onset of melting from the DSC endotherm was determined to be 330° C. for the PTFE. The tube was recovered/heat shrunk over a mandrel of 6.53 mm at 350° C. for 10 minutes. A DMA temperature sweep was carried out at 1 Hz on a rectangular sample cut from the circumference of the original composite tube and slit open. The results are shown in Table 1.

### Example 4

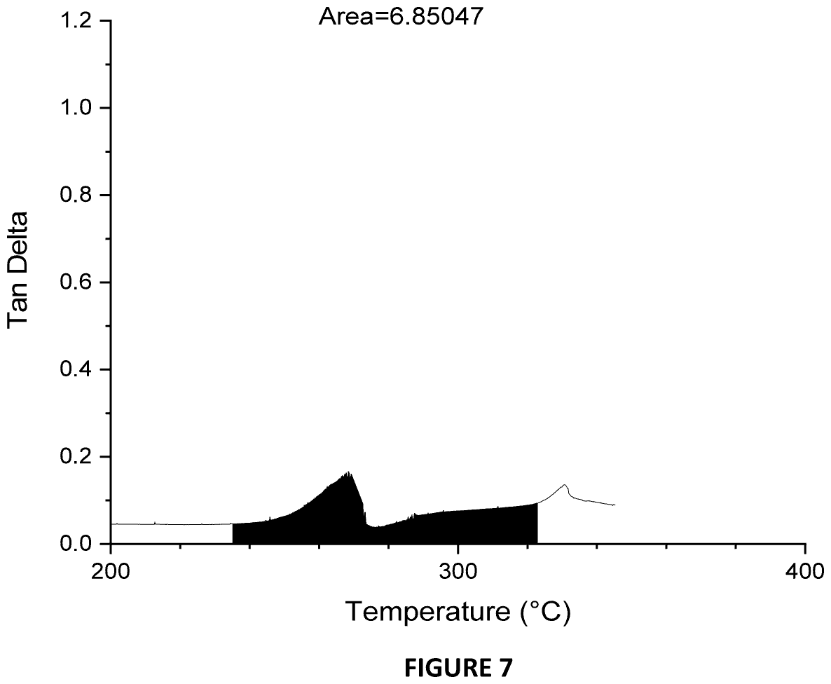

A dual layer heat shrink tube with initial outer diameter of 4.22 mm was manufactured with an outer layer of PTFE (with a wall thickness of 260 μm) expanded between 33° and 350° C. at a ratio of 2:1 and an inner layer of FEP (with a wall thickness of 200 μm). The onset of melting from the DSC endotherm was determined to be 235° C. for the FEP. The onset of melting from the DSC endotherm was determined to be 323° C. for the PTFE. The tube was recovered/heat shrunk over a mandrel of 1.90 mm at 350° C. for 10 minutes. A DMA temperature sweep was carried out at 1 Hz on a rectangular sample cut from the circumference of the original composite tube and slit open. The results are shown in Table 1 and in FIG. 7.

### Comparative Example 4

A dual layer heat shrink tube with an initial outer diameter of 7.88 mm was manufactured with an outer tube of PTFE (200 μm wall thickness) expanded between 33° and 350° C. at a ratio of 2:1, and an inner layer of PFA filled with 4% w/w carbon black (51 μm wall thickness). The onset of melting from the DSC endotherm was determined to be 290° C. for the filled PFA. The onset of melting from the DSC endotherm was determined to be 323° C. for the PTFE. The dual layer heat shrink tube was recovered/heat shrunk over a mandrel of 4.17 mm at 350° C. for 10 minutes. A DMA temperature sweep was carried out at 1 Hz on a rectangular sample cut from the circumference of the original composite tube and slit open. The results are shown in Table 1.

### Example 5

A dual layer heat shrink tube with an initial outer diameter of 7.88 mm was manufactured with an outer tube of PTFE (200 μm wall thickness) expanded between 33° and 350° C. at a ratio of 2:1, and an inner layer of FEP filled with 15% w/w carbon black (358 μm wall thickness). The onset of melting from the DSC endotherm was determined to be 265° C. for the FEP blend. The onset of melting from the DSC endotherm was determined to be 330° C. for the PTFE. The tube was recovered/heat shrunk over a mandrel of 2.28 mm at 350° C. for 10 minutes. A DMA temperature sweep was carried out at 1 Hz on a rectangular sample cut from the circumference of the original composite tube and slit open. The results are shown in Table 1.

### Example 6

A dual layer heat shrink tube with an initial outer diameter of 4.19 mm was manufactured with an outer tube of FEP (435 μm wall thickness) expanded between 18° and 220° C. at a ratio of 1.6:1, and an inner layer of EFEP (254 μm wall thickness). The onset of melting from the DSC endotherm was determined to be 175° C. for the EFEP. The onset of melting from the DSC endotherm was determined to be 245° C. for the FEP. The tube was recovered/heat shrunk over a mandrel of 1.42 mm at 350° C. for 10 minutes. A DMA temperature sweep was carried out at 1 Hz on a rectangular sample cut from the circumference of the original composite tube and slit open. The results are shown in Table 1.

### Example 7

A dual layer heat shrink tube with an initial outer diameter of 5.95 mm was manufactured with an outer tube of PTFE (284 μm wall thickness) expanded between 33° and 350° C. at a ratio of 2:1, and an inner layer of PVDF (445 μm wall thickness). The onset of melting from the DSC endotherm was determined to be 160° C. for the PVDF. The onset of melting from the DSC endotherm was determined to be 323° C. for the PTFE. The tube was recovered/heat shrunk over a mandrel of 2.22 mm at 350° C. for 10 minutes. A DMA temperature sweep was carried out at 1 Hz on a rectangular sample cut from the circumference of the original composite tube and slit open. The results are shown in Table 1.

### Example 8

A dual layer heat shrink tube with an initial outer diameter of 6.29 mm was manufactured with an outer tube of FEP (262 μm wall thickness) expanded between 18° and 220° C. at a ratio of 1.3:1, and an inner layer of PVDF (445 μm wall thickness). The onset of melting from the DSC endotherm was determined to be 160° C. for the PVDF. The onset of melting from the DSC endotherm was determined to be 240° C. for the FEP. The tube was recovered/heat shrunk over a mandrel of 2.51 mm at 220° C. for 10 minutes. Once removed from the oven, it was discovered that the outer FEP tube could easily be removed from the inner PVDF tube. A DMA temperature sweep was carried out at 1 Hz on a rectangular sample cut from the circumference of the original composite tube and slit open. The results are shown in Table 1.

TABLE 1
Summary of Test Results

| | Temp. | Area | Δ % Length | Over- | |
|---|---|---|---|---|---|
| Exam- | Range for | under tan | +15%, | flow | Overall |
| ple | Integration | δ curve, ° C. | −2% | >5 mm | Rating |
| Comp. 1 | 265-323° C. | 19.5 | −4.6 | Yes | Poor |
| Comp. 2 | 160-323° C. | 59.6 | −10.4 | No | Poor |
| Ex. 1 | 265-323° C. | 7.6 | 9.9 | No | Good |
| Ex. 2 | 265-323° C. | 6.9 | 3.5 | No | Good |
| Ex. 3 | 265-323° C. | 2.5 | 7.3 | No | Good |
| Comp. 3 | 235-323° C. | 13.0 | −2.4 | No | Poor |
| Ex. 4 | 235-323° C. | 6.9 | 6.6 | No | Good |
| Comp. 4 | 290-323° C. | 1.0 | 20.8 | No | Poor |
| Ex. 5 | 240-323° C. | 7.7 | 6.5 | No | Good |
| Ex. 6 | 175-243° C. | 4.3 | 10.4 | No | Good |
| Ex. 7 | 160-323° C. | 10.7 | 7.3 | No | Good |
| Ex. 8 | 160-240° C. | 9.1 | 1.0 | No | Good |

### Example 9

A commercially available poly (ether-block-amide) (PEBA) resin (Arkema, Inc, PEBAX® 5533 SA 01 MED) was obtained in pellet form and dried in a membrane dryer at 167° F. overnight to ensure the resin moisture content was less than 0.15% by weight. The dried PEBA resin was placed into a resin hopper dryer to prevent re-absorption of moisture before and during extrusion. A commercially available maleic anhydride grafted linear low density polyethylene (LLDPE-g-MA) resin (Arkema, Inc, OREVAC® 18300M) was obtained in pellet form and placed in the resin hopper of a second single screw extruder. The dried PEBA pellets were melted and conveyed to a multilayer head by utilizing a single screw extruder having a barrel diameter of ¾″, a screw rotation of 5-6 rpm, and barrel zone temperatures of 380-410° F. The LLDPE-g-MA pellets were melted and conveyed to a multilayer head by utilizing a second single screw extruder having a barrel diameter of ¾″, a screw rotation of 6-7 rpm, and barrel zone temperatures of 380-410° F. The multilayer head was configured to have the PEBA as the outer layer and the LLDPE-g-MA to be the inner layer of a multilayer tube and utilized a die temperature of 390-405° F., and an annular die set that provided a draw down ratio (DDR) of 12-14. After exiting the annular die set, the multilayer input tube was passed through a chilled water bath to sufficiently quench the tubing and set the final tubular dimensions. A multilayer input tube having an average inner diameter of 0.065″, an average total wall thickness of 0.005″, and an average layer ratio of 50:50 obtained using this process.

The prepared multilayer input tube was then expanded by pressurizing the inner diameter of the tube with compressed air as the input tube resided within a stainless steel hypodermic tube (also referred to herein as a “hypodermic tube” or “hypo tube” or just “hypo”) and a forced air heating element (also referred to herein as a “thermal nozzle”) passed along the length of the hypodermic tube. The hypo tube serves as an expansion die and is used to restrict expansion of the multilayer input tube to a specified expanded diameter. The processing parameters of expansion air pressure applied to the ID of the multilayer input tubing, temperature of the pressurized expansion air applied to the ID of the multilayer input tubing, forced air heating element air temperature, forced air heating element air flowrate, forced air heating element traversal rate were all adjusted to give a non-crosslinked multilayer PEBA heat shrink tube with adhesive inner layer according to the present disclosure.

In particular, the multilayer heat shrink input tube of Example 9 was expanded using a forced air heating element air temperature of 255-265° F., an expansion air pressure of 35 psi, forced air heating element air flowrate of 40 liters/minute, and a forced air heating element traversal rate of 1.2 mm/s.

### Catheter Examples

### Example 10

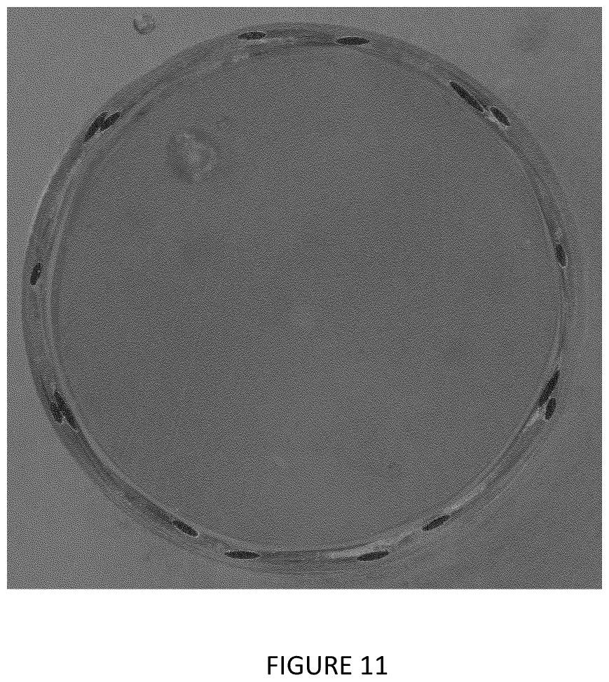

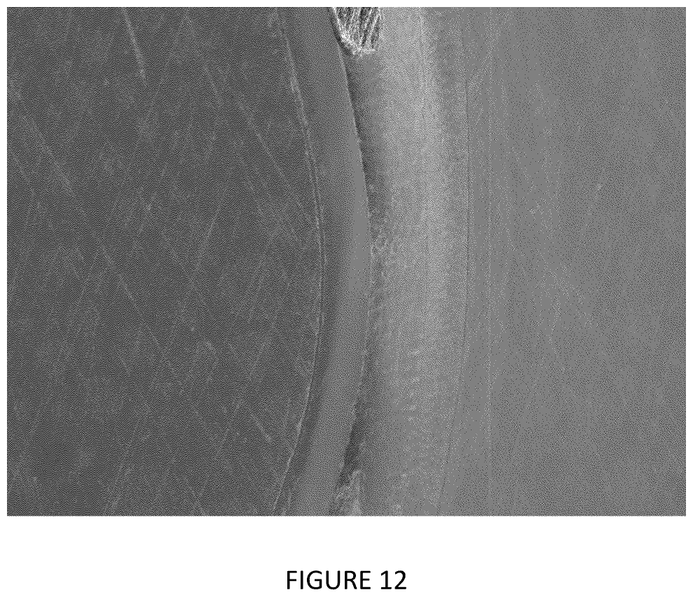

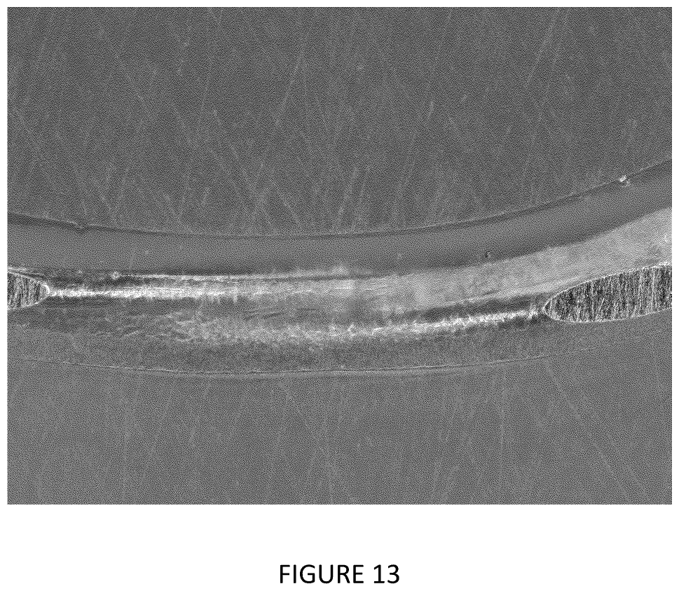

A PTFE tube with a 0.110″ average OD and a 0.030″ average wall was inserted into the ID of an OD etched PTFE liner consisting of a 0.115″ average ID and a 0.0015″ average wall thickness. The PTFE tube acts as a support mandrel for the OD etched PTFE liner. A Steeger USA medical catheter braider fitted with 16 carriers supporting 0.002″ nominal OD stainless steel braid wire was used to apply full braid pattern with an average of 80 picks per inch to the outer surface of the supported OD etched PTFE liner. The multilayer heat shrink tube from Example 9 was then slid over top of the braided surface of the supported OD etched PTFE liner. This assembly (i.e., the over braided supported OD etched PTFE liner and multilayer heat shrink tube of Example 9) was suspended vertically and heated in a Beahm 815A vertical laminator equipped with a thermal nozzle set at 310° F. and utilizing a thermal nozzle traverse rate of 0.2 mm/s to provide a braid reinforced catheter shaft with an outer jacket comprising non-crosslinked PEBA and maleated polyolefin layers. After cooling, the 0.110″ OD PTFE support tube was removed from the ID of completed build. A section was cut from the completed build and cast in 2-part acrylic resin. Once the acrylic resin had cured the specimen was sectioned with an abrasive sectioning saw and then polished to yield a mounted sample of the completed build. The mounted sample was examined using a Keyence VHX-5000 digital microscope to determine if the non-crosslinked inner layer of the multilayer heat shrink tube flowed within the interstices of the braid reinforcement and made adequate contact with the outer surface of the inner liner during the heating process. Images captured during this examination are shown in FIG. 11, FIG. 12, and FIG. 13.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A multilayer heat shrink tubing comprising:
an outer layer comprising non-crosslinked PEBA; and
an inner layer comprising maleated polyolefin,
wherein the maleated polyolefin has a melt temperature that is lower than the melt temperature of the outer layer.

2. The multilayer heat shrink tubing of claim 1 wherein the heat shrink tubing has a recovery ratio (RR) greater than about 1.05:1.

3. The multilayer heat shrink tubing of claim 1, wherein the outer layer consists essentially of the non-crosslinked PEBA.

4. The multilayer heat shrink tubing of claim 1, wherein the inner layer consists essentially of the maleated polyolefin.

5. The multilayer heat shrink tubing of claim 1, wherein the maleated polyolefin comprises one or more of maleic anhydride grafted polyethylene, maleic anhydride grafted polypropylene, and maleic anhydride grafted ethylene vinyl acetate copolymer.

6. The multilayer heat shrink tubing of claim 1, wherein the maleated polyolefin comprises maleic anhydride grafted linear low density polyethylene.

7. The multilayer heat shrink tubing of claim 1, wherein the weight ratio of the outer layer to the inner layer is between 99:1 and 1:99.

8. The multilayer heat shrink tubing of claim 1, wherein the weight ratio of the outer layer to the inner layer is between 95:5 and 5:95.

9. The multilayer heat shrink tubing of claim 1, wherein the weight ratio of the outer layer to the inner layer is between 90:10 and 10:90.

10. The multilayer heat shrink tubing of claim 1, wherein the weight ratio of the outer layer to the inner layer is between 75:25 and 25:75.

11. The multilayer heat shrink tubing of claim 1, wherein the RR is greater than about 1.10:1 and/or is reducible in ID by about 9.1%.

12. The multilayer heat shrink tubing of claim 1, wherein the RR is greater than about 1.2:1 and/or reducible in ID by about 16.7%.

13. The multilayer heat shrink tubing of claim 1, wherein the RR is greater than about 1.3:1 and/or reducible in ID by about 23.1%.

14. The multilayer heat shrink tubing of claim 1, wherein the RR is greater than about 1.4:1 and/or reducible in ID by about 28.6%.

15. The multilayer heat shrink tubing of claim 1, wherein the RR is greater than about 1.5:1 and/or reducible in ID by about 33.3%.

16. The multilayer heat shrink tubing of claim 1, wherein the RR is greater than about 1.6:1 and/or reducible in ID by about 37.5%.

17. The multilayer heat shrink tubing of claim 1, wherein a durometer hardness measurement of the outer layer of a flat specimen fabricated by melt pressing the heat shrink tubing in expanded form is about 20 to 80 Shore D.
